(19) United States
(12) Patent Application Publication
(10) Pub. No.: US 2018/0128804 A1
(43) Pub. Date: May 10, 2018

(54) ESSENTIAL NUTRIENT RATIO DETERMINATION

(71) Applicant: Waters Technologies Corporation, Milford, MA (US)

(72) Inventors: Giuseppe Astarita, Hopkinton, MA (US); Donald Mason, Haverhill, MA (US); Michael Balogh, Rehoboth, MA (US)

(21) Appl. No.: 15/577,761

(22) PCT Filed: May 26, 2016
(86) PCT No.: PCT/US2016/034288

§ 371 Date: Nov. 28, 2017

Related U.S. Application Data
(60) Provisional application No. 62/168,187, filed on May 29, 2015.

(51) Int. Cl. G01N 33/03; G01N 33/92

(52) U.S. Cl. CPC G01N 33/03; G01N 27/62; G01N 33/92

(57) ABSTRACT

The present disclosure relates generally to methods and apparatus for determining essential nutrients and the ratio of essential nutrients in a sample. In particular, the present disclosure relates to the use of surface desorption ionization-mass spectrometry methods and apparatus to assay essential nutrients and ratios thereof, e.g., omega-6/omega-3 ratios.

### CROSS-REFERENCE TO RELATED APPLICATIONS

This application claims the benefit of and priority to U.S. Provisional Patent Application No. 62/168,187, filed on May 29, 2015, the entire contents of which is incorporated herein by reference.

### TECHNICAL FIELD

The present disclosure relates generally to methods and apparatus for determining essential nutrients and the ratio of essential nutrients in a sample. In particular, the present disclosure relates to the use of surface desorption ionization-mass spectrometry methods and apparatus to assay essential nutrients and ratios thereof, e.g., omega-6/omega-3 ratios.

### BACKGROUND OF THE INVENTION

Omega-6 fatty acids and omega-3 fatty acids are polyunsaturated fatty acids (PUFAs). These fatty acids are in a class of lipids essential to humans and need to be absorbed through diet. Both are required to maintain certain bodily functions and to keep humans healthy. While both classes are fatty acids, each class has different roles and effects in the body.

Omega-6 fatty acids are ingested mainly from plant oils such as corn oil, soybean oil, and sunflower oil, as well as from nuts and seeds. Omega-6 fatty acids can aid in a variety of ways, such as in reducing the symptoms of diabetic neuropathy, rheumatoid arthritis, allergies and high blood pressure. Additionally, they can ease the symptoms of menopause, multiple sclerosis, ADHD, eczema, menstrual pain and breast cancer. Omega-3 fatty acids are ingested mainly from fatty fish such as salmon, mackerel, and tuna, as well as from walnuts and flaxseed in lesser amounts. Omega-3 fatty acids can also aid in a variety of ways, such as the reduction of heart disease and in the function of the brain and in normal growth development. They can also stimulate hair and skin growth, reduce some types of inflammation, and reduce blood pressure and high cholesterol.

Both omega-6 fatty acids and omega-3 fatty acids in the body should be in balance to maintain optimum health. The overall balance between omega-6 fatty acids and omega-3 fatty acids can affect the above conditions as well as modulate many other biological processes including the relaxation and contraction of smooth muscle tissue, blood coagulation and, most notably, inflammation.

Current test methods for determining fatty acids require laborious and time-consuming procedures, which make them unsuitable for screening applications. For example, gas chromatography-mass spectrometry (GC-MS) has been traditionally the technique of choice. Analysis by GC-MS, however, requires a multi-step procedure for the hydrolysis and derivatization of the fatty acids to fatty acid methyl esters, and a chromatographic separation. Alternatively, liquid chromatography-tandem mass spectrometry (LC-MS) has been used and allows for the direct measurement of both free and esterified fatty acids without the need for hydrolysis or derivatization. Yet, LC-MS still requires the labor intensive and time consuming chromatographic separation step. Supercritical fluid chromatography-mass spectrometry and other similar techniques has also been used, but these techniques also suffer from the same requirement. Furthermore, any detailed spatial distribution of these species on a sample surface is unavailable using traditional sample preparation and extraction protocols.

The present disclosure relates to methods and apparatus for both screening an imaging essential nutrients, including fatty acids, in samples which are less time consuming and resource intensive.

### SUMMARY OF THE INVENTION

The present disclosure relates generally to methods and apparatus for determining essential nutrients and the ratio of essential nutrients in a sample. In particular, the present disclosure relates to the use of surface desorption ionization-mass spectrometry methods and apparatus to assay essential nutrients and ratios thereof, e.g., omega-6/omega-3 ratios. The ratio and the method of determining the ratio can be incorporated into, or used as, a diagnostic test.

In one embodiment the present disclosure relates to a method of determining the ratio of omega-6 polyunsaturated fatty acids to omega-3 polyunsaturated fatty acids including generating sample ions from a sample containing omega-6 polyunsaturated fatty acids and omega-3 polyunsaturated fatty acids using a surface desorption ionization source, receiving the ions into a mass spectrometer, identifying the omega-6 polyunsaturated fatty acids and omega-3 polyunsaturated fatty acids, and calculating the ratio of omega-6 polyunsaturated fatty acids to omega-3 polyunsaturated fatty acids present in the sample. The surface desorption ionization source can be, or preformed using, an atmospheric solid analysis probe, direct analysis in real time, rapid evaporative ionization mass spectrometry, desorption electrospray ionization, matrix assisted laser desorption ionization or nanostructure and initiated mass spectrometry. The method can screen samples, e.g., a diet, food, supplements, dosage forms, and can be performed in a short time, such as in less than 24 hours.

In another embodiment, the present disclosure relates to a method of determining the spatial distribution of omega-6 polyunsaturated fatty acids and omega-3 polyunsaturated fatty acids on a sample surface including generating sample ions from a first location on a sample containing omega-6 polyunsaturated fatty acids and omega-3 polyunsaturated fatty acids using an ionizing source, receiving the ions into a mass spectrometer, determining the omega-6 polyunsaturated fatty acids and omega-3 polyunsaturated fatty acids present in the sample at the first location, and repeating these steps on a plurality of locations.

In another embodiment, the present disclosure relates to a method of treating a fatty acid deficiency, or a disorder or condition associated with a fatty acid imbalance or deficiency, including determining the ratio of omega-6 polyunsaturated fatty acids to omega-3 polyunsaturated fatty acids in a patient suffering from a fatty acid deficiency, etc., determining a healthy ratio of omega-6 polyunsaturated fatty acids to omega-3 polyunsaturated fatty acids in the patient, and administering a diet, a food, a supplement or a dosage form to the patient, wherein the administered composition comprises a ratio of omega-6 polyunsaturated fatty acids to omega-3 polyunsaturated fatty acids capable of adjusting the patient's ratio of omega-6 polyunsaturated fatty acids to omega-3 polyunsaturated fatty acids closer to the healthy ratio.

In another embodiment, the present disclosure relates to a diet, food, supplement, a microalgae, or dosage form label including the ratio of omega-6 polyunsaturated fatty acids to omega-3 polyunsaturated fatty acids in the diet, food, supplement or dosage form.

The methods and apparatus of the present disclosure provide several advantages over the prior art. The present disclosure provides a quick and simple method of assessing lipid profiles and ratios between various fatty acid species. Such assessments can be indicative of the health status of living organisms or the quality of a diet, food, supplement or dosage form. Fatty acid composition affects the physiology of living cells. By assessing the amount of and/or alterations in fatty acid profiles a wide range of conditions or pathologies in various organisms can be monitored, reduced, treated or prevented. The methods and apparatus can be used to monitor the health status and well-being of individuals and populations.

The use of desorption ionization techniques and mass spectrometry also provides a level of description beyond the pure measure of fatty acid concentration. The present disclosure can be used for real-time, rapid, in-situ screening of various materials including food, plant and animal tissue. The present disclosure can also be performed without an internal standard or pre-calibrations, and can distinguish between the various fatty acid species.

### BRIEF DESCRIPTION OF THE DRAWINGS

The foregoing and other features and advantages provided by the present disclosure will be more fully understood from the following description of exemplary embodiments when read together with the accompanying drawings, in which:

FIG. 1 shows an overview of omega-6 and omega-3 fatty acids present in food. The ratio of omega-6 and omega-3 fatty acids in a diet can vary from about 1 (within historically normal ratios) to 50 (extremely high ratio common with Western diets). The ratio of omega-6 and omega-3 fatty acids in different oils is also shown and can vary greatly.

FIG. 2 shows an overview of various risk factors for sudden cardiac death, including overall omega-3 fatty acids.

FIG. 3 shows an exemplary food label listing Supplemental Facts including omega-3 fatty acids. Standard food labeling does not include these supplemental facts. The US FDA has published guidance for the development of new omega-3 pharmaceutical products to be sold in the US. This includes documentation of the effect of the product on plasma levels of bound and unbound (e.g., esterified and non-esterified) EPA and/or DHA. Current method to measure unbound fatty acids require chromatographic techniques such as thin layer chromatography or solid phase extraction which can be time consuming and expensive.

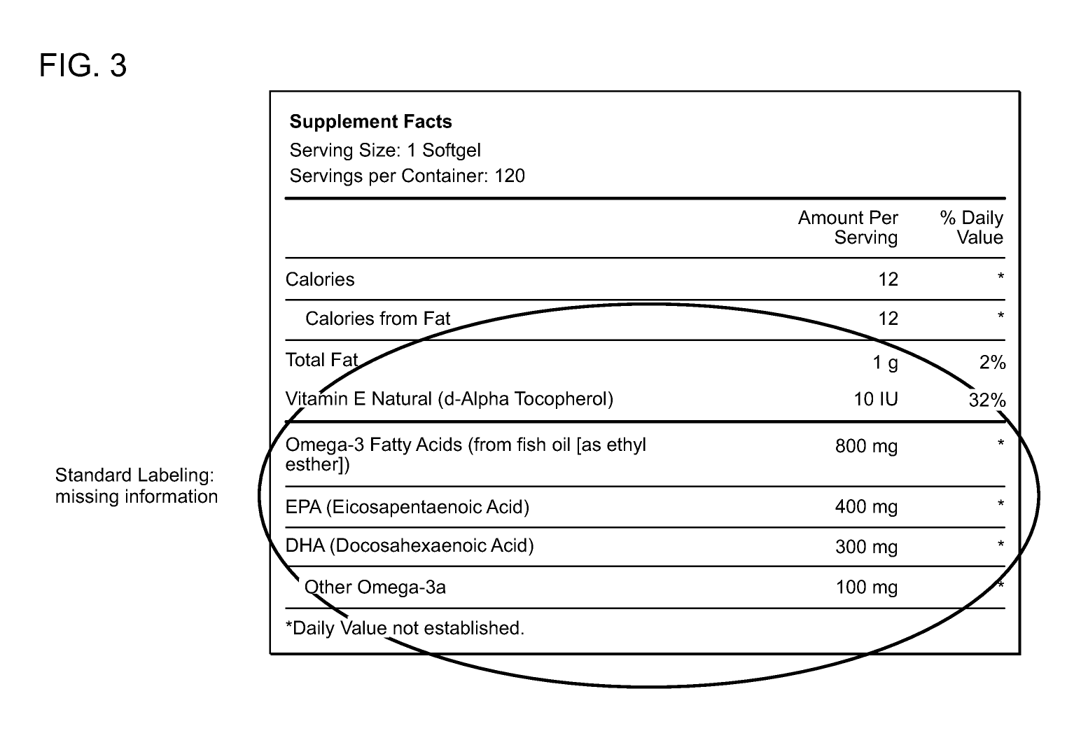

FIG. 4 shows an illustration of the unmet need for lipid screening, e.g., the ratio of omega-6 fatty acids to omega-3 fatty acids. For example, individuals can have a sample taken, e.g., blood, and have it screened using a fast and simple diagnostic tool (“box”), e.g., surface desorption ionization-mass spectrometry as described herein, to determine the lipid composition of the sample and whether the individual suffers from any lipid disorder or imbalance. The individual can be treated with a diet, food, supplement or dosage form to change or correct the disorder or imbalance. The individual can be regularly re-screened to monitor the effects the treatment.

FIG. 5 shows an exemplary embodiment of the present disclosure including a sample preparation device for use with direct-analysis in real time (i.e., DART) and a single quadrupole mass spectrometer. For example, blood samples can be spotted on the mesh sample areas (indicated by the arrows).

FIG. 6 shows exemplary mass spectrometry results of fatty acids from three different whole blood spots tested using direct-analysis in real time and a single quadrupole mass spectrometer. The samples contain an internal standard and fatty acids. Analysis of the three different blood drops shows reproducible results in terms of the response of the internal standard, fatty acid content and ratios.

FIG. 7 shows exemplary mass spectrometry results of fatty acids from three different fish oil spots tested using direct-analysis in real time and a single quadrupole mass spectrometer. The samples contain an internal standard and fatty acids. Analysis of the three different fish oil drops shows reproducible results in terms of the response of the internal standard, fatty acid content and ratios.

FIG. 8 shows exemplary groups and classes of fatty acids that can be monitored and determined using the present disclosure and various calculations that can be used to determine the various indexes and ratios, including saturated fatty acid index, monounsaturated fatty acid index, PUFA index, omega-3 index, omega-3 highly unsaturated fatty acid index, peroxidation index, unsaturation index and desaturation index.

FIG. 9 shows an exemplary list of other classes of metabolites that can be similarly monitored and determined (e.g., absolute content and ratios) using the present disclosure.

FIG. 10 shows two exemplary portable, small, real-time analysis systems (A and B) of the present disclosure.

### DETAILED DESCRIPTION

The present disclosure relates to methods and apparatus for determining essential nutrients and the ratio of essential nutrients in a sample. In particular, the present disclosure relates to the use of surface desorption ionization-mass spectrometry methods and apparatus to assay essential nutrients and ratios thereof, e.g., omega-6/omega-3 ratios.

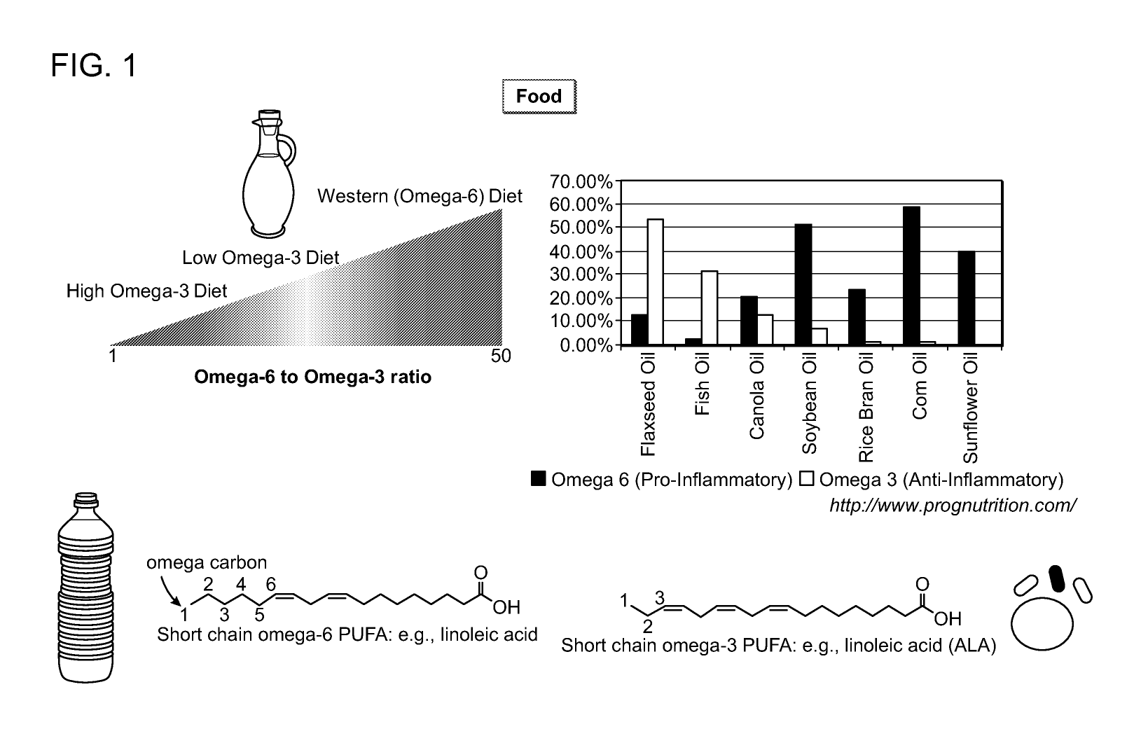

Omega fatty acids are polyunsaturated fatty acids characterized by a carboxylic group, an aliphatic chain, and multiple double bonds. They are named according to the position of the first double bond in the carbon chain, starting from the terminal carbon atom of the molecule (called the “omega carbon” because omega is the last letter of the Greek alphabet). See FIG. 1 for exemplary fatty acids.

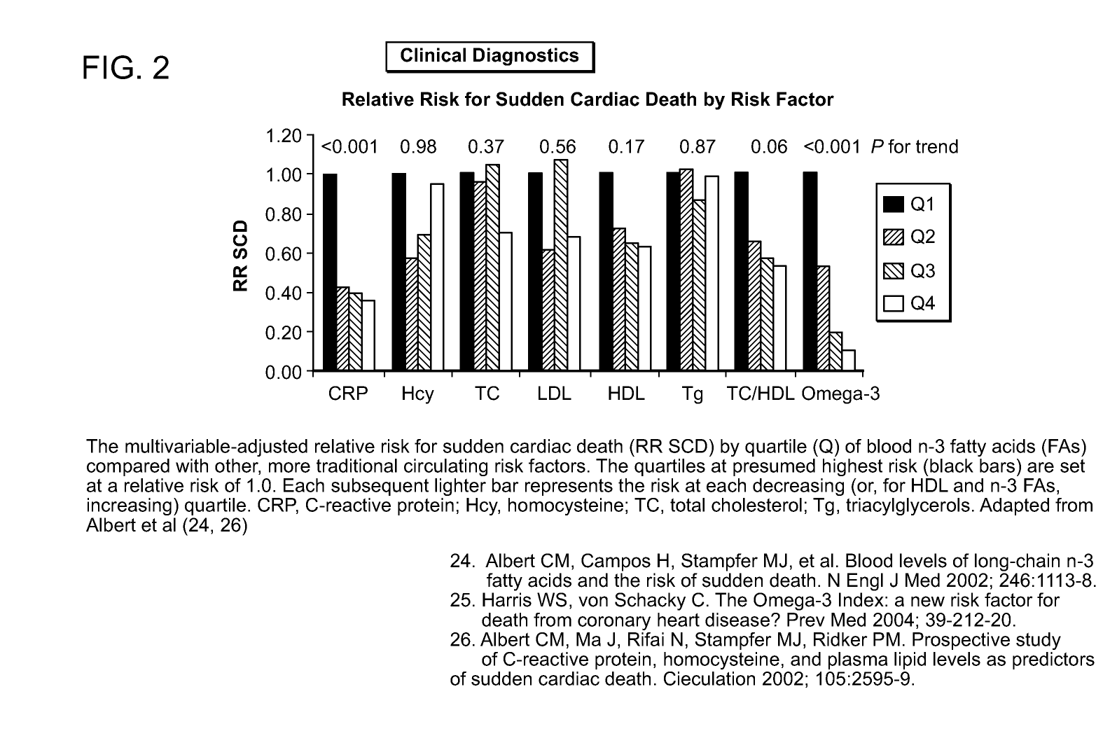

In one aspect, the importance of fatty acids to human health includes the overall balance between omega-6 and omega-3 fatty acids. This balance, or ratio, can modulate many biological processes including the relaxation and contraction of smooth muscle tissue, blood coagulation and inflammation. Some long-chain omega-3 fatty acids are found to be particularly enriched in the brain and retina, playing a major role in cognition and vision. FIG. 2 shows an overview of the relative risk factors for sudden cardiac death, including omega-3 fatty acids, which play a role. The ratio of omega-6 fatty acids to omega-3 fatty acids (o6/o3) is also believed impact this, and other conditions, as described herein.

Within each omega family, there are also subclass distinctions based on chain length, e.g., short-chain and long-chain fatty acids. The human body cannot manufacture short-chain polyunsaturated fatty acids and must rely entirely on dietary intake for these essential nutrients. Long-chain polyunsaturated fatty acids, on the other hand, can be made by the body starting from a shorter chain or can be absorbed directly through diet. Short-chain omega-3 fatty acids are abundant in foods as alpha-linolenic acid (ALA). In particular, ALA is present at high levels in leafy green vegetables and flaxseeds. The most abundant dietary long-chain omega-3 fatty acids are eicosapentanoic acid (EPA) and docosahexaenoic acid (DHA), which are present in oily fish and fish oil supplements. Omega-6 fatty acids mainly include the short-chain linoleic acid (LA) and to a lesser extent the long-chain arachidonic acids (ARA), which are abundant in vegetables oils, such as corn, soybean, safflower and sunflower oils.

As shown in FIG. 1, most Western diets are deficient in omega-3 fatty acids and abundant in omega-6 fatty acids. Current nutritional research shows that a diet enriched in omega-3 fatty acids offers health benefits and anti-inflammatory properties, whereas an excess of omega-6 fatty acids might contribute to the pathogenesis of many chronic inflammatory diseases, including cardiovascular and autoimmune diseases. Consequently, the present disclosure relates to the development of a rapid and inexpensive assay for screening nutrients, e.g., omega-6 and omega-3 fatty acids, not only for labeling foodstuffs but also to assess nutritional deficiencies or imbalances. The present disclosure is also related to personalized nutritional interventions aimed at balancing select nutrients, e.g., omega-6 and omega-3 fatty acids, to improve overall health.

In one embodiment, the present invention relates to a method of determining the ratio of omega-6 polyunsaturated fatty acids to omega-3 polyunsaturated fatty acids including generating sample ions from a sample containing omega-6 polyunsaturated fatty acids and omega-3 polyunsaturated fatty acids using a surface desorption ionization source, receiving the ions into a mass spectrometer, and identifying the omega-6 polyunsaturated fatty acids and omega-3 polyunsaturated fatty acids, and calculating the ratio of omega-6 polyunsaturated fatty acids to omega-3 polyunsaturated fatty acids present in the sample.

The sample can be any sample containing two or more analytes of interest, or classes of analytes of interest, that can be effectively tested using the surface desorption ionization-mass spectrometry methods and apparatus described herein. In one embodiment, the sample is a diet, a food, a supplement, a dosage form or a biological sample. For example, the method and apparatus of the present disclosure can be used to assess the properties, e.g., inflammatory and nutritional status, of a biological sample including whole blood, plasma and red blood cells.

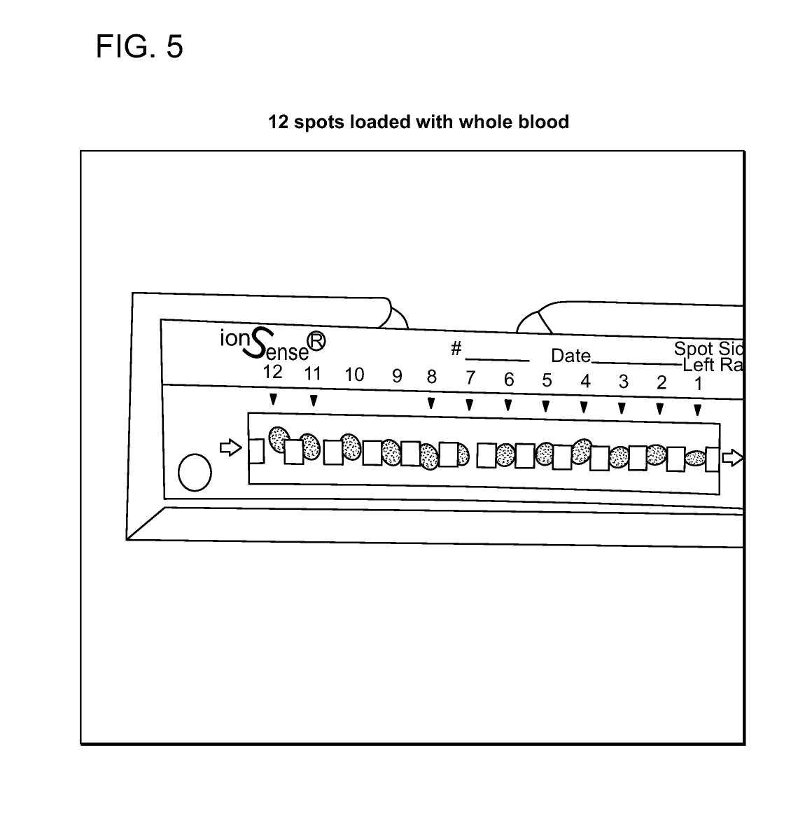

The sample can be analyzed with no substantial preparation, such as filtering, extraction, isolation or combinations thereof. The sample can be analyzed neat, or with no sample preparation. For example, a sample or samples can be swiped on glass capillaries and held, placed or otherwise introduced to the ionization source, e.g., held in a metastable gas beam between the direct analysis in real time ion source and a mass spectrometer detector. See FIG. 5 for an exemplary sample mesh capable of holding the sample in the ionization source. In one embodiment, the sample preparation is simple such that the sample can be a biological sample, e.g., a dried blood drop spotted on a slide or grid. The biological sample can also be blood in solution, as well as skin, sebum, saliva, plasma, serum, urine, hair, tissue biopsies, etc.

The sample can also be associated with a diet. The sample can be a food or foods, a supplement or supplements. The food or supplement(s) can be in any form, e.g., solid or liquid. For example, the sample can be an edible oil or butter, e.g., olive oil, fish oil, coconut oil, canola oil, safflower oil, almond butter, peanut butter, etc. The sample can also be taken from green leafy plants, fish, microalgae or algae. The sample can be a beverage, milk or human breast milk.

The sample can also be associated with a dosage form to treat a condition or imbalance. The dosage form can be in any form, e.g., tablet, capsule, pill, film, liquid, etc. Depending on the dosage form, the sample can be prepared by neat or by altering the dosage form to access the sample. For example, the sample can be a capsule containing a specific dosage of omega-6 fatty acids and omega-3 fatty acids. The sample preparation can include removing a portion of the contents from inside the capsule. In one embodiment, the measure can be used to monitor inflammation status, as proxy of inflammation, also after surgery or pharmacological treatment, as diagnostic or prognostic or predictive marker of inflammation or marker of response or toxicity or exposure.

The sample ions can be generated using any desorption ionization (DI) source or technique capable of effectively sampling analytes of interest, or classes of analytes of interest, from a sample for introduction into a mass spectrometer. The desorption ionization source or technique can also be any capable of real-time, rapid in-situ testing of solid or liquid samples. In one embodiment, the desorption ionization source is a surface desorption ionization source or technique.

In desorption ionization, the ionization process can begin by irradiating, or otherwise exposing, a defined spot on a sample, e.g., solid sample, using a focused energy source. The energy source can be an excitatory beam such as a laser, ions, charged, solvent droplets or heated gas containing metastable ions. Upon impact, the sample's surface releases a vapor of ionized molecules that can be directed into a mass spectrometer. Alternatively, acoustic or thermal desorption can initiate the ionization process.

In one embodiment, the analysis of fatty acids using a surface desorption ionization-mass spectrometry system is provided. Fatty acids are particularly suited for surface desorption ionization because fatty acids can be in high abundance in biological and food samples, and they can ionize well in negative mode under DI conditions.

The surface desorption ionization source can operate by a technique selected from the group consisting of electrospray ionization, nano-electrospray ionization, matrix-assisted laser desorption ionization, atmospheric pressure chemical ionization, desorption electrospray ionization, atmospheric pressure dielectric barrier discharge ionization, atmospheric pressure low temperature plasma desorption ionization, laser-assisted electrospray ionization, and electrospray-assisted laser desorption ionization.

In particular, the surface desorption ionization source can operate by a technique selected from the group consisting of atmospheric solid analysis probe (i.e., ASAP), direct analysis in real time (DART), rapid evaporative ionization mass spectrometry (REIMS), desorption electrospray ionization (DESI), matrix assisted laser desorption ionization (MALDI), nanostructure and initiated mass spectrometry (NIMS).

The desorption ionization source can small and have a small footprint. The desorption ionization source can also be suitable or compatible with ambient mass spectrometry, e.g., a mass spectrometer operating at or near atmospheric pressure. In one embodiment, the desorption ionization source or technique is DART, ASAP, REIMS or DESI. These ionization sources can be small and compatible with ambient mass spectrometry.

Direct Analysis in Real Time is an atmospheric pressure ion source that can instantaneously ionizes gases, liquids or solids in open air under ambient conditions. It is an ambient ionization technique that does not require sample preparation, so solid or liquid materials can be analyzed by mass spectrometry in their native state. Ionization can take place directly on the sample surface. Liquids can be analyzed by, for example, dipping an object (such as a glass rod) into the liquid sample and then presenting it to the DART ion source. Vapors can be introduced directly into the DART gas stream.

Atmospheric Solids Analysis Probe is an atmospheric pressure ion source that can directly analyze samples using an atmospheric pressure ionization (API) source. The ASAP probe can analyze solid, liquid, tissue, or material samples. In ASAP, vaporization of a sample can occur when it is exposed to a hot desolvation gas, e.g., nitrogen, from an probe, e.g., an electrospray ionization or atmospheric pressure chemical ionization probe.

Rapid Evaporative Ionization Mass Spectrometry (REIMS) is an ionization technique that can be used as a source for direct analysis of samples by mass spectrometry. REIMS is an atmospheric pressure ion source that can ionize gases, liquids or solids in open air under ambient conditions. The REIMS ionization source can be a probe that can be used to remotely test the samples. See U.S. Patent Publication No. 2012/0156712, the disclosure of which is incorporated herein in its entirety.

Desorption electrospray ionization (DESI) is an ambient ionization technique that can be used in mass spectrometry for chemical analysis. It is an atmospheric pressure ion source that ionizes gases, liquids and solids in open air under ambient conditions. DESI is a combination of electrospray (ESI) and desorption (DI) ionization methods. Ionization can take place by directing an electrically charged mist to a sample surface. The electrospray mist can be attracted to the surface by applying a voltage on the sample or sample holder. After ionization, the ions can travel through air into the atmospheric pressure interface which can be connected to a mass spectrometer.

Thermal desorption ionization can be used as the ionization mechanism. The sample, and biological components, can be exposed to different temperatures to induce ionization. See U.S. Patent Publication No. 2013/0299688, the disclosure of which is incorporated herein in its entirety.

In some embodiments, the energy or temperature of the ionization source may not be sufficiently high to efficiently ionize a representative sample. For example, the sample may contain fatty acids having different properties, such as different volatilities. At a certain energy level or temperature, some fatty acids may be ionized more readily than others, which can create a bias in the ratio at that energy level or temperature. In one embodiment, the present disclosure includes a step of determining a sufficient energy level (e.g., temperature in thermal desorption) to ionize a representative sample of all components, analytes of interest, or classes of analytes of interest. For example, the energy level can be tested at increasing values until the intensities or ratio of intensities for the analytes of interest stabilize at a constant value indicative of a representative sampling of analytes.

In another embodiment, the energy level of the ionization source can be sufficiently high to ionize the free fatty acids. The energy level can be relatively high because fatty acids are stable and do not fragment easily. Yet, fatty acids can also be present a sample as component of a complex lipid, e.g., esterified. The energy level of the ionization source can be sufficiently high to ionize the free fatty acids, but sufficiently low to prevent release and ionization of the non-free fatty acids. For example, the surface desorption ionization source can operate at a sufficiently energy to efficiently ionize a representative sample of omega-6 polyunsaturated fatty acids to omega-3 polyunsaturated fatty acids present in the sample, but not saturated fatty acids or complexed and esterified fatty acids. The energy can be calibrated to ionize complex lipid containing fatty acids.

The method can also be robust such that the sampling does not exhaust the components, analytes of interest or classes of analytes of interest, e.g., omega acids, in the sample. The ionization process can involve a short, e.g., less than about 10 seconds, 9, 8, 7, 6, 5, 4, 3, 2, 1, 0.5, 0.2 or about 0.1 seconds, exposure of the ionization source to the sample.

The sample ions can be received or introduced to a mass spectrometer by any means or technique capable of effectively introducing ions into a mass spectrometer that can allow for real-time, rapid in-situ testing of solid or liquid samples. For example, the ions can be introduced under ambient conditions.

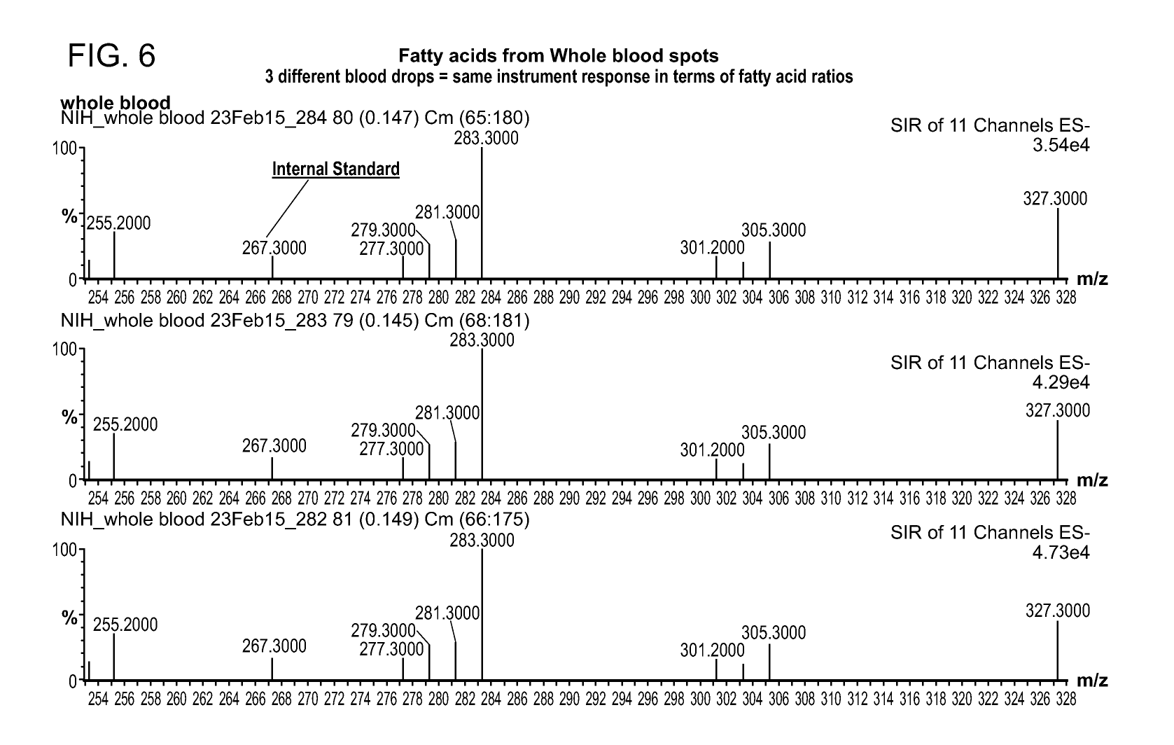

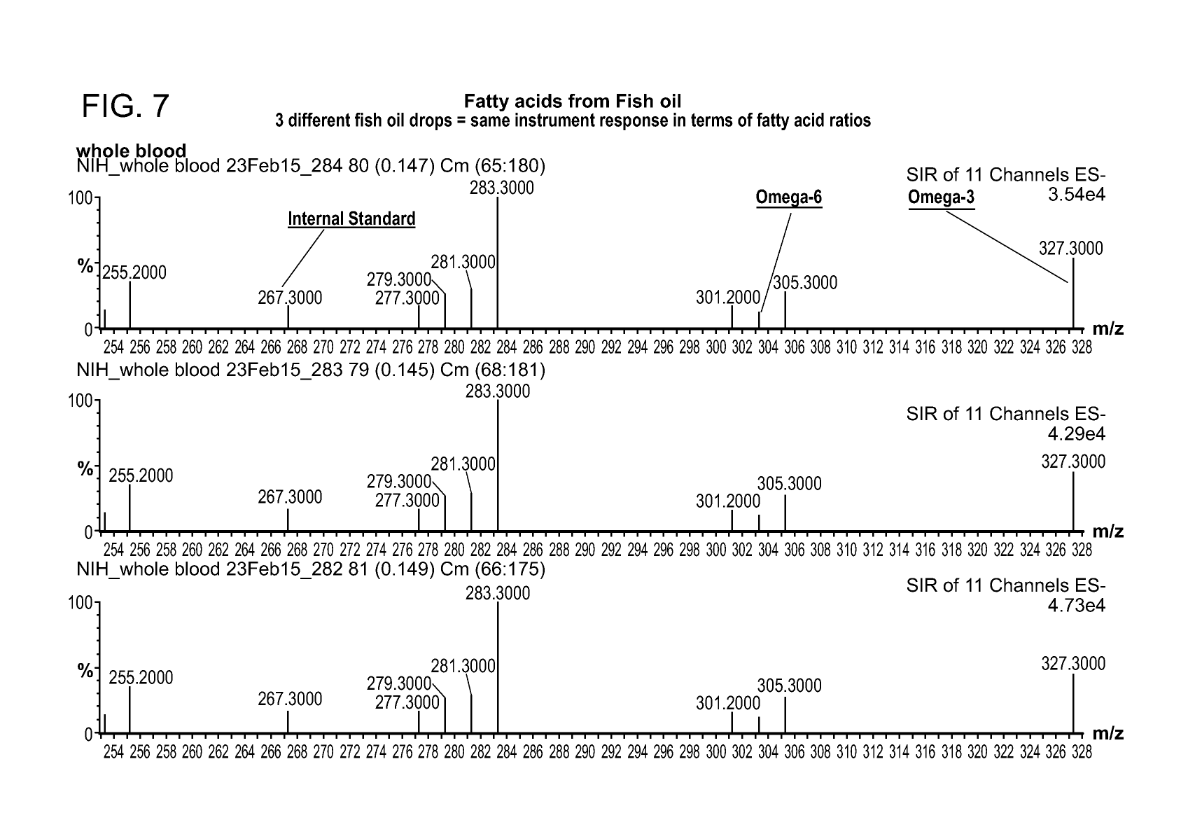

The mass spectrometer can be any mass spectrometer capable of receiving the sample ions, of producing accurate mass measurements, and of identifying sample analytes of interest. See FIGS. 6 and 7 for exemplary mass spectrometry results for blood samples and fish oil. The mass spectrometer can be a quadrupole mass spectrometer, portable ion trap mass spectrometer, time of flight mass spectrometer, Fourier transform ion cyclotron resonance mass spectrometry, orbi trap or ion mobility spectrometer. For example, the mass spectrometer can be a single quadrupole QDa® detector, e.g., a DART-QDa®.

The analytes of interest can be analyzed by selection reaction monitoring in a quadrupole instrument. Selection reaction monitor involves pre-selection of a list of ions of interest or extracted from full scan accurate mass spectra, in which no ion is preselected but the quadrupole is scanned along all the mass range selected (e.g., 50-100 m/z).

The mass spectrometer can be operated in positive or negative mode. In one embodiment, the mass spectrometer is operated in negative mode under desorption ionization conditions. Fatty acid ionize particularly well in negative mode. The coupling of a mass spectrometer, e.g., a single quadrupole device, with desorption ionization can also allow for the direct analysis of fatty acids as a function of peak intensity or as a ratio between peaks or groups of peaks. The ratio of fatty acids can be used to normalize for variation in instrument settings and sampling. For example, a variation in intensity of one fatty acid(s) is compensated by an equivalent variation in another fatty acid(s). Their ratio can be used to normalize for difference between samples. For example, a selected number of fatty acids with unique m/z can be monitored, including arachidonic acid (mlz 303.3 AA, omega-6), docosahexaenoic acid (mlz 327.3, DHA, omega-3), eicosapentaenoic acid (mlz 301.3 EPA, omega-3), linoleic acid (mlz 277.3 LA, omega-6) and alpha linoleic acid (mlz 279.3 ALA, omega-3).

The ratio(s) of analytes of interest, or classes of analytes of interest, can be calculated from the mass spectrometry results. The ratio can be calculated using the intensity of the peaks. The ratio can be calculated with or without the use of an internal standard. The ratio can be a simple ratio of the intensities of the mass signals. The use of internal standard can provide semi-quantification after correcting for any isotopic contribution to the signal. For example, internal standards can be used to normalize the concentration of the fatty acids in the samples to obtain a more quantitative measurement.

In some embodiments, the analytes of interest, e.g., fatty acids, can be derivatized or tagged before DI-MS analysis. MS/MS analysis of the tagged analytes can then be performed. For example, charge-reversal derivatization of fatty acids can be performed wherein the carboxylic acids are converted into cationic derivatives with quaternary amines. Detection by ESI can be improved. Also, electron capture atmospheric pressure chemical ionization can be performed on analytes that have been tagged with an electron-capturing group such as the pentafluorobenzyl moiety. Detection by APCI can be improved.

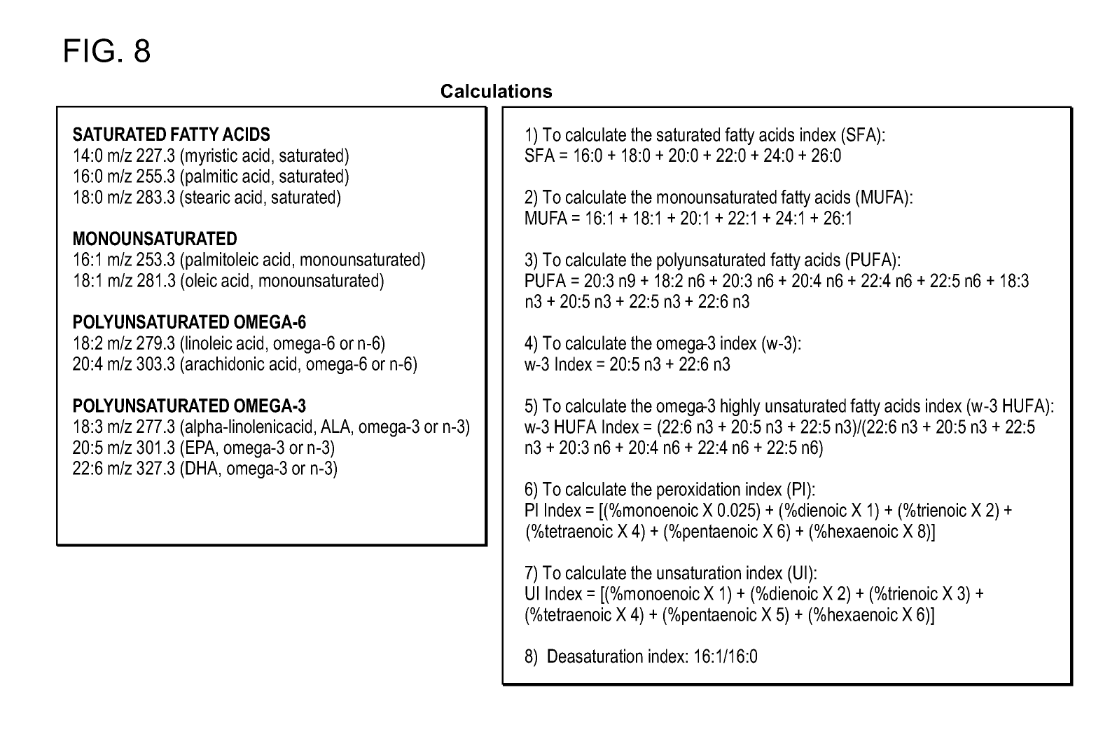

FIG. 8 shows an exemplary embodiment of the types of calculations that can be performed to determine the analytes of interest, or classes of analytes of interest (e.g., saturated fatty acids, monounsaturated, etc.) and ratios thereof. For example, exemplary ratios of omega-6 fatty acids to omega-3 fatty acids can include:

arachidonic acid/(eicosapentanoic acid+docosapentanoic acid)

arachidonic acid/(eicosapentanoic acid+docosahexaenoic acid)

arachidonic acid+linoleic acid/(eicosapentanoic acid+docosapentanoic acid+linolenic acid), and

alpha-linolenic acid/linoleic acid

An exemplary ratio that can be used as an index of essential fatty acid deficiency includes triene/tetraene ratio=eicosatrienoic acid/arachidonic acid.

In another embodiment, the measurement of ratio can be extended to between a compound(s) of interest and an internal standard(s), or as a percent composition of an overall class of lipids (e.g., % DHA). For example, hexose, glucose, acylcarnitines, amino acids, complex lipids, etc. can be monitored using this apparatus and similar ratiometric methods.

Additional omega nutritional scores can be calculated using the methods and apparatus of the present disclosure. For example, an Omega 3-6 Balance Score (O3-6BS) for an individual food item can be calculated as follows.

O3-6BS=(mg short3−mg short6)/Cal+7×(mg long 3−mg long6)/Cal

The resulting score characterizes the balance of essential fatty acids in each food item independent of any other foods that might be eaten during the day.

Dietary 18-carbon polyunsaturated fatty acids (PUFA) maintain the proportions of 20- and 22-carbon highly unsaturated fatty acid (HUFA) hormone precursors that are accumulated in tissues. Knowing the metabolic interaction can provide insight for a preventive nutrition strategy based on the health risk assessment biomarker, e.g., % n-6 in tissue HUFA. Dietary omega-3 fatty acids are “n-3”, dietary omega-6 fatty acids are “n-6”, and dietary omega-9 fatty acids are “n-9” The following equation describes the % n-6 in tissue HUFA.

% n-6 in HUFA=[100×(n-6HUFA)]/[n-3HUFA+n-6HUFA+n-9HUFA]

Recognizing that many researchers have found that dietary HUFA affect tissue HUFA proportions more than dietary PUFA do, an empirical scaling factor can be used to generate daily menu balance (dmb) values over a range from approximately −10 to +10.

dmb=(en % short3−en % short6)+(factor)×(en % long 3−en % long6)

where omega-6 PUFA (“short 6”; e.g., 18:2 and 18:3), omega-3 PUFA (“short 3”; e.g., 18:3 and 18:4), omega-6 HUFA (“long 6”; e.g., 20:3, 20:4, 22:4 and 22:5) and omega-3 HUFA (“long 3”; e.g., 20:5, 22:5 and 22:6), calorie (“Cal”). The daily intake of the fatty acids categories can be expressed as a percentage of the overall daily food energy (en %). See Lands et al., Nutrition & Metabolism 2012, 9:46 “Using 3-6 differences in essential fatty acids rather than 3/6 ratios gives useful food balance scores,” which is incorporated herein by reference.

In exemplary embodiments, the present disclosure relates to a particular class of fatty acids, e.g., the polyunsaturated fatty acids omega-3 and omega-6 and their metabolites. Additional analytes of interest, e.g., additional fatty acids, can also be analyzed, such as by selection reaction monitoring in a quadrupole instrument or extracted from full scan accurate mass spectra, including 14:0 m/z 227.3 (myristic acid, saturated), 16:0 m/z 255.3 (palmitic acid, saturated), 16:1 m/z 253.3 (palmitoleic acid, monounsaturated), 18:0 m/z 283.3 (stearic acid, saturated), 18:1 m/z 281 .3 (oleic acid, monounsaturated), 18:2 m/z 279.3 (linoleic acid, omega-6 or n-6), 18:3 m/z 277.3 (alpha-linolenic acid, ALA, omega-3 or n-3), 20:4 m/z 303.3 (arachidonic acid, omega-6 or n-6), 20:5 m/z 301.3 (EPA, omega-3 or n-3), 22:6 m/z 327.3 (DHA, omega-3 or n-3). In addition, selection reaction monitoring of long chain fatty acids (markers of peroxisomal disorders) 22:0, 22:1, 24:0, 24:1 can also be performed.

In general, the concentration of each analyte, e.g., lipid, can be calculated using the following equation:

Area of unknown lipid/(Area of internal standard)*(Concentration of internal standard)/weight of tissue expressed in grams or volume of liquid (e.g., biofluid).

In another embodiment, the present disclosure also applies to the detection of metabolites derived by fatty acids. For example, oxidation of polyunsaturated fatty acids through enzymatic or non-enzymatic free radical-mediated reactions can yield an array of lipid metabolites including eicosanoids, octadecanoids, docosanoids and related species. In mammals, these oxygenated PUFA mediators can play prominent roles in the physiological and pathological regulation of many key biological processes in the cardiovascular, renal, reproductive and other systems including their pivotal contribution to inflammation.

The method of the present disclosure can determine the ratio of components, analytes of interest, or classes of analytes of interest, in a shorter time that methodology of the prior art. The method can determine the ratio within 10 seconds, 20, 30, 40, 50 or 60 seconds, 2 minutes, 3, 4, 5, 10, 20, 30, 40, 50 or 60 minutes, or 1.5 hours, 2, 3, 4, 5, 6, 7, 8, 9, 10, 11, 12 or 24 hours. These values can also be used to define a range, such as between about 10 minutes and about 60 minutes. In another embodiment, the present disclosure can determine the ratio without sending a sample to a laboratory for analysis. The methodology can be used as a point of care test.

The present disclosure can determine the ratio without extraction, hydrolysis, filtration, derivatization, chromatographic separation (e.g., GC-FID) or combinations thereof. The prior art methodology involves one or more of these steps and can take hours to complete, e.g., at least about 2 hours. The method of the present disclosure can reduce the analysis time by about 10%, 20, 30, 40, 50, 60, 70, 80, 90, 100, 200, 300, 500, or about 1000%. These values can also be used to define a range, such as between about 20% and about 50%.

In another embodiment, the present disclosure relates to a method of determining the spatial distribution of omega-6 polyunsaturated fatty acids and omega-3 polyunsaturated fatty acids on a sample surface including generating sample ions from a first location on a sample containing omega-6 polyunsaturated fatty acids and omega-3 polyunsaturated fatty acids using an ionizing source, receiving the ions into a mass spectrometer, determining the omega-6 polyunsaturated fatty acids and omega-3 polyunsaturated fatty acids present in the sample at the first location, and repeating these steps on a plurality of locations. The ratio of omega-6 polyunsaturated fatty acids to omega-3 polyunsaturated fatty acids present can also be determined in the sample at each location.

The first location on a sample surface can be any location. The additional locations on the sample surface, e.g., the plurality of locations, can be any other locations on the sample surface. In one embodiment, the locations are all separate locations on the sample surface. The analysis at each location can be performed by either direct sampling from the sample surface by the desorption ionization source, or from samples removed from the plurality of locations.

The distance between adjacent locations can vary based on the level of detail and resolution desired for the spatial distribution analysis. To provide sufficiently detailed spatial distribution analysis, the average distance between adjacent locations can be less than about 100 mm, 90, 80, 70, 60, 50, 40, 30, 20, 10, 50, 20, 1, or about 0.5 mm. These values can also be used to define a range, such as between about 10 and 1 mm.

In another embodiment, the present disclosure relates to a method of treating a fatty acid deficiency, or a disorder or condition associated with a fatty acid imbalance or deficiency, including determining the ratio of omega-6 polyunsaturated fatty acids to omega-3 polyunsaturated fatty acids in a patient suffering from a fatty acid deficiency, etc. determining a healthy ratio of omega-6 polyunsaturated fatty acids to omega-3 polyunsaturated fatty acids in the patient, and administering a diet, a food, a supplement or a dosage form to the patient, wherein the administered composition comprises a ratio of omega-6 polyunsaturated fatty acids to omega-3 polyunsaturated fatty acids capable of adjusting the patient's ratio of omega-6 polyunsaturated fatty acids to omega-3 polyunsaturated fatty acids closer to the healthy ratio.

A fatty acid deficiency, or a disorder or condition associated with a fatty acid imbalance or deficiency can by any disorder related to lipid or fatty acid imbalance or deficiency including a patient having an unhealthy ratio of omega-6 polyunsaturated fatty acids to omega-3 polyunsaturated fatty acids. An unhealthy ratio is one that negatively impacts or affects the health of the patient. In some embodiments, a healthy ratio of omega-6 polyunsaturated fatty acids to omega-3 polyunsaturated fatty acids can be about 1:1 to about 3:1. An healthy ratio can be any ratio less than about 3:1, 2.9:1, 2.8:1, 2.7:1, 2.6:1, 2.5:1, 2.4:1, 2.3:1, 2.2:1 or about 2.1:1. An healthy ratio can also be any ratio greater that about 1.1:1, 1.2:1, 1.3:1, 1.4:1, 1.5:1, 1.6:1, 1.7;1, 1.8:1, 1.9:1 or about 2.0:1. These values can also be used to define a range, such about 2.5:1 to about 1.5:1.

Depending on the individual, some ratios can be healthy for some and be unhealthy for others. For different individual, there can be overlap in the ranges of health to unhealthy ratios. An unhealthy ratio can be any ratio less than about 1:1, 0.9:1, 0.8:1, 0.7:1, 0.6:1, 0.5:1, 0.4:1, 0.3:1, 0.2:1 or about 0.1:1. These values can also be used to define a range, such about 1:1 to about 0.5:1. An unhealthy ratio can also be any ratio greater that about 1:3, 1:4, 1:5, 1:6, 1:7, 1:8, 1:9, 1:10, 1:20, 1:30, 1:40, or about 1:50. These values can also be used to define a range, such about 4:1 to about 20:1.

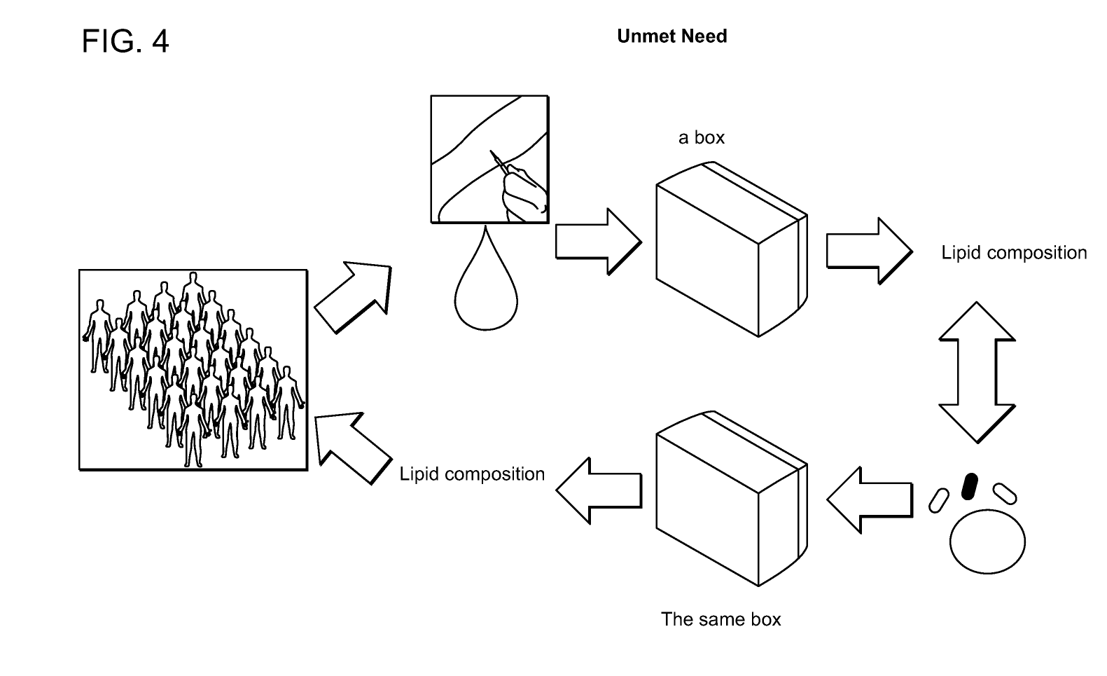

The present disclosure also relates to a diagnostic test or screening method to determine the ratio of omega 6 fatty acids to omega 3 fatty acids in a subject (e.g., whole blood, plasma, red blood cells, etc.). The ratio of two or more other biologically significant components can also be determined. The ratio information can be used by the person, a medical professional, etc. to devise a treatment plan to correct or adjust the ratio. See FIG. 4. The therapy or treatment can include administering a food, supplement or diet to adjust the ratio to a pre-determined value or to adjust the value to a newer value that is about 5%, 10%, 20%, 30%, 40% or about 50% greater or less than originally determined.

A diet, a food, a supplement or a dosage form having a ratio of omega-6 polyunsaturated fatty acids to omega-3 polyunsaturated fatty acids capable of adjusting the patient's ratio of omega-6 polyunsaturated fatty acids to omega-3 polyunsaturated fatty acids closer to the healthy ratio can be administered. The diet, food, supplement or dosage form can contain a ratio of omega-6 polyunsaturated fatty acids to omega-3 polyunsaturated fatty acids from about 99:1 to about 1:99.

The diet, food, supplement or dosage form can have various amounts of nutrients, e.g., omega-6 and omega-3 polyunsaturated fatty acids. The diet, food, supplement or dosage form can have at least about 10%, 20%, 30%, 40%, 50%, 60%, 70%, 80%, 90%, 100%, 150%, or 200% of the recommended daily allowance of lipids, or fatty acids, omega-6 fatty acids, omega-3 fatty acids or both omega-6 and omega-3 fatty acids. These values can also be used to define a range, such about 10% to about 50%.

The diet, food, supplement or dosage form can have at least about 100 mg, 200 mg, 300 mg, 400 mg, 500 mg, 600 mg, 700 mg, 800 mg, 900 mg, 1 g, 1.1 g, 1.2 g, 1.3 g, 1.4 g, 1.5 g, 2 g, 2.5 g, 3 g, 4 g, or about 5 g of lipids, or fatty acids, omega-6 fatty acids, omega-3 fatty acids or both omega-6 and omega-3 fatty acids. These values can also be used to define a range, such about 500 mg and about 2 g. The diet, food, supplement or dosage form can also have a label including the ratio of specific nutrients, e.g., omega-6 polyunsaturated fatty acids to omega-3 polyunsaturated fatty acids, in the diet, food, supplement or dosage form. The label claim ratio can be determined by the method and apparatus of the present disclosure.

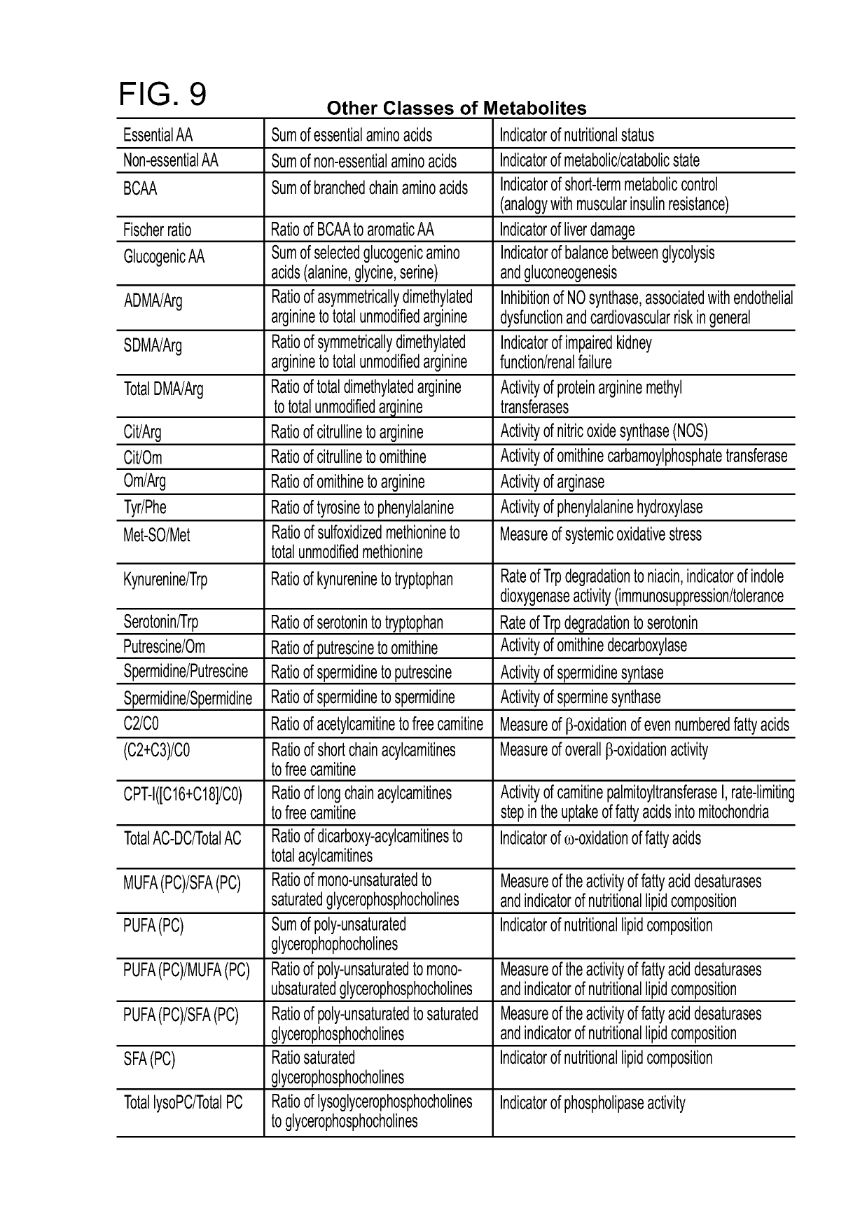

The deficiency, disorder or condition described herein, as well as the treatment and diagnostic test or screening methods can be associated with other nutrients or analytes as also described herein. The present disclosure can be used to test for the ratio of other biological substances in a patient, food, supplements, etc. The ratio can also be a measure of nutritional unbalance or proxy of enzymatic activities and function. By monitoring the ratio, for example in blood or serum, the tissue omega index or ratio can be predicted as a function of diet, treatment, etc. The ratio can also be indicative of the following disorders or conditions—the risk of suicide, mood disorders, depressive symptoms, perception of stress or happiness. For example, the present disclosure can be used to test for amino acids, immunoglobulins, combination therapy serum levels, hormones, biofuels, insulin/sugar, etc. FIG. 9 lists additional classes of metabolites that can be monitored, identified and used to develop a healthy range or ratio to evaluate a diet, food, supplement or dosage form.

The disclosures of all cited references including publications, patents, and patent applications are expressly incorporated herein by reference in their entirety.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention is further defined in the following Examples. It should be understood that these Examples, while indicating preferred embodiments of the invention, are given by way of illustration only.

### EXAMPLES

### Example 1

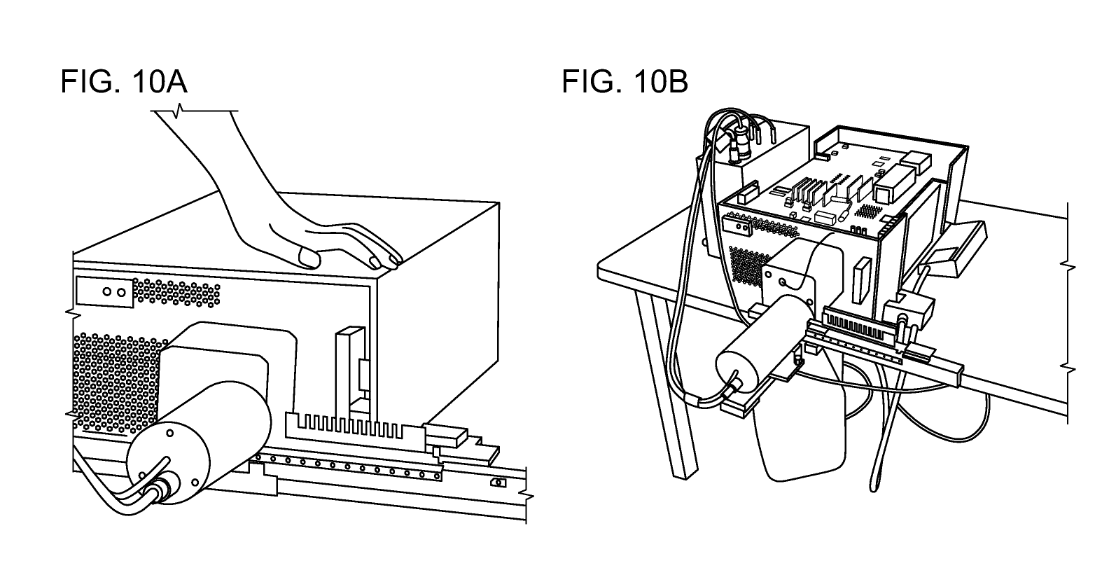

A portable, small system for real-time analysis of various samples, e.g., biological samples, was prepared. The system includes surface desorption ionization, e.g., direct analysis in real time, coupled to a single quadrupole mass spectrometer, e.g., ACQUITY® QDa® Mass Detector. FIG. 10 shows two pictures of the portable, small, real-time analysis system. The portable, small design allows the system to be a point of care device (e.g., for use in doctor's office, clinics, wellness centers, laboratories, customer self service stations).

Essential nutrients, e.g., omega-6 and omega-3 fatty acids, in a sample were analyzed using a single quadrupole mass spectrometer equipped with direct analysis in real time desorption ionization source. No chromatographic separation was required. Blood samples were tested by placing a single drop of blood on the direct analysis in real time interface with the single quadrupole mass spectrometer. The sample was obtained by applying a lancet on the side of an alcohol wiped finger and blood was collected and placed on a sample card. The blood was allowed to dry for about 15 minutes. Twelve samples were collected and placed on individual spots on the card with no cross-contamination. The twelve sample positions were loaded manually (can also be done automatically) including standards for quantitation. Alternatively, samples can be collected using tweezers, etx. Also, a system to collect, or collect and store, blood drops can include one or more antioxidants or mixture thereof to apply to the blood collected, the blood drops or paper. The antioxidant can prolong the viability of the blood or blood samples for analysis. The amount of antioxidant can be less than about 10 wt % of the blood collected or blood drop sample, or less than about 9%, 8, 7, 6, 5, 4, 3, 2, 1, 0.5, 0.2, 0.1, 0.05 or 0.01 wt % of the blood collected or blood drop sample. These values can be used to define a range, such as about 2 to about 0.1 wt %. Viability can be increased by about 10%, 20, 30, 40, 50, 100, 150, 200, 500 or 1000% compared to a sample not containing at least one antioxidant or mixture thereof. These values can be used to define a range, such as about 20 to about 100 wt %.

The analyses were conducted using a direct analysis in real time (DART, IonSense, Mass., USA) source coupled with a single quadrupole mass spectrometer (Acquity® QDa®, Waters Corporation, Milford, Mass., USA). The acquisition time was about 5-10 seconds, Ionization DART +ve and −ve; Cone voltage 20.0 V; Source temp. 120.0° C.; DART temp. 50 to 450° C.

A complete fatty acid profile was provided in real time, without sample preparation. A bioinformatics solution was used to translate the intensity ratios in health status and well-being measures and generate reports associated with nutritional recommendations.

1. A method of determining the ratio of omega-6 polyunsaturated fatty acids to omega-3 polyunsaturated fatty acids comprising:
(i) generating sample ions from a sample containing omega-6 polyunsaturated fatty acids and omega-3 polyunsaturated fatty acids using a surface desorption ionization source;
(ii) receiving the ions into a mass spectrometer;
(iii) identifying the omega-6 polyunsaturated fatty acids and omega-3 polyunsaturated fatty acids; and
(iv) calculating the ratio of omega-6 polyunsaturated fatty acids to omega-3 polyunsaturated fatty acids present in the sample.

2. The method of claim 1 wherein the surface desorption ionization source operates by a technique selected from the group consisting of electrospray ionization, nano-electrospray ionization, matrix-assisted laser desorption ionization, atmospheric pressure chemical ionization, desorption electrospray ionization, atmospheric pressure dielectric barrier discharge ionization, atmospheric pressure low temperature plasma desorption ionization, laser-assisted electrospray ionization, direct analysis in real time, atmospheric solids analysis probe technique, rapid evaporative ionization mass spectrometry and electrospray-assisted laser desorption ionization.

3. The method of claim 1 wherein the surface desorption ionization source operates by a technique selected from the group consisting of atmospheric solid analysis probe, direct analysis in real time, rapid evaporative ionization mass spectrometry, desorption electrospray ionization, matrix assisted laser desorption ionization or nanostructure and initiated mass spectrometry.

4. The method of claim 1 wherein the surface desorption ionization source operates at a sufficiently high energy to efficiently ionize a representative sample of omega-6 polyunsaturated fatty acids to omega-3 polyunsaturated fatty acids present in the sample.

5. The method of claim 1 wherein the mass spectrometer is a quadrupole mass spectrometer, time of flight mass spectrometer, ion trap mass spectrometer or Fourier transform ion cyclotron resonance mass spectrometry.

6. The method of claim 1 wherein steps (i)-(iii) are performed in less than 5 minutes.

7. The method of claim 1 wherein the sample is a diet, a food, a supplement, a dosage form or a biological sample.

8. A method of determining the spatial distribution of omega-6 polyunsaturated fatty acids and omega-3 polyunsaturated fatty acids on a sample surface comprising:
(i) generating sample ions from a first location on a sample containing omega-6 polyunsaturated fatty acids and omega-3 polyunsaturated fatty acids using an ionizing source;
(ii) receiving the ions into a mass spectrometer;
(iii) determining the omega-6 polyunsaturated fatty acids and omega-3 polyunsaturated fatty acids present in the sample at the first location, and
(iv) repeating (i)-(iii) on a plurality of locations.

9. The method of claim 8 further comprising calculating the ratio of omega-6 polyunsaturated fatty acids to omega-3 polyunsaturated fatty acids present in the sample at each location.

10. A method of treating a fatty acid deficiency or fatty acid related condition comprising:
(i) determining the ratio of omega-6 polyunsaturated fatty acids to omega-3 polyunsaturated fatty acids in a patient suffering from a fatty acid deficiency or fatty acid related condition;
(ii) determining a healthy ratio of omega-6 polyunsaturated fatty acids to omega-3 polyunsaturated fatty acids in the patient; and
(ii) administering a diet, a food, a supplement or a dosage form to the patient, wherein the administered composition comprises a ratio of omega-6 polyunsaturated fatty acids to omega-3 polyunsaturated fatty acids capable of adjusting the patient's ratio of omega-6 polyunsaturated fatty acids to omega-3 polyunsaturated fatty acids closer to the healthy ratio.

11. (canceled)

12. (canceled)
